(19) United States
(12) Patent Application Publication
(10) Pub. No.: US 2004/0122427 A1
(43) Pub. Date: Jun. 24, 2004

(54) Apparatus and method for restoring biomechanical function to a motion segment unit of the spine

(76) Inventor: David C. Holmes, Boca Raton, FL (US)

Correspondence Address: WATOV & KIPNES, P.C., P.O. Box 247, Princeton Junction, NY, 08550, US

(21) Appl. No.: 10/683,505

(22) Filed: Oct. 10, 2003

Related U.S. Application Data
(60) Provisional application No. 60/417,610, filed on Oct. 10, 2002.

(51) Int. Cl. A61B 17/56

(52) U.S. Cl. 606/61

(57) ABSTRACT

A method of restoring stability to an unstable motion segment unit of the spine in which pre and post decompression measurements of at least one characteristic of the targeted motion segment unit, or combination of motion segment units, are compared with a data bank of measurements of the same characteristics of known motion segment units, then matched with suitable implantable spinal assist device to identify a suitable device for correcting instability of the targeted motion segment unit. The implant selected for restabilization of any one patient's spine joint will be in part, a function of an objective, intraoperative stiffness measurement, which in turn, is a function of the integrity of a patient's surviving spine joint tissues at this time-post decompression.

### RELATED APPLICATION

This application relies for priority on U.S. Provisional Patent Application Serial No. 60/417,610 filed Oct. 10, 2002.

### FIELD OF THE INVENTION

The present invention is generally directed to an apparatus and method for measuring instability of a motion segment unit of the spine and for restoring normal or near normal biomechanical function to the motion segment unit. The present invention enables a surgeon to quantitatively determine whether and to what extent the spine has lost the ability to function under normal physiological loads and to precisely correct any such lost ability through the selective identification and insertion of at least one implantable spinal assist device.

### BACKGROUND OF THE INVENTION

It is well known that back pain is one of the most frequently occurring and expensive disabling ailments, especially for patients in the 30-50 year old age bracket. Although back pain syndrome is a very common occurrence, for many years its diagnosis was very difficult to realize. The vertebral column (spine) is a biomechanical structure composed primarily of ligaments, muscles, vertebrae and intervertebral discs. The biomechanical functions of the spine include (1) support of the body (trunk and appendages), which involves the transfer of the weight and the bending moments of the head, trunk and arms to the pelvis and legs, (2) complex physiologic motion between these body parts and (3) protection of the spinal cord and the nerve roots.

The major regions of the spine are the cervical, thoracic, lumbar and sacral. The vertebrae increase in size and mass from the cervical to the lumbar regions. The increase and size of the vertebrae is directly related to an increased capacity for supporting larger loads. The lumbar region is therefore the major load supporter of the spine. However, this increase in load supporting capacity is paralleled by a decrease in flexibility. Because the lumbar region supports heavier loads than other regions of the spine, the lumbar trunk (low back structure) is more susceptible to strain and hence low-back pain.

The spine is comprised of different levels known as motion segment units or spine joints. The lumbar spine is comprised of five motion segment units. The motion segment unit is the smallest component of the spine that exhibits kinematic behavior similar to that of the whole spine. The motion segment unit is capable of discrete movements such as flexion, extension, lateral bending and translation. The components of each motion segment unit include two adjacent vertebrae and their apophyseal joints, the invertebral disc and the connecting ligamentous tissue.

Many causes of back pain are attributed to the instability of a motion segment unit. Segmental instability is defined as “the loss of ability of the spine under physiologic loads to maintain relationships between vertebrae in such a way that there is neither damage nor subsequent irritation to the spinal cord or nerve routes, and, in addition, there is no development of incapacitating deformity or pain due to structural changes”. In other words, instability is an abnormal response to applied loads characterized by motion in the motion segment unit beyond normal constraints. Excess motion can be abnormal in quality (i.e. abnormal coupling patterns) or in quantity (i.e. abnormal increased motion) or both. Excess motion results in damage to the nerve routes, the spinal cord and other spinal structures.

The underlining causes of the structural changes in the motion segment unit leading to stability are trauma, degeneration, aging, disease (tumor, infection, etc.), surgery or combination thereof. It is known that a mechanically unstable motion segment unit can originate due to degeneration of the annulus fibrosis and nucleus pulposus. A degenerate nucleus causes disc space narrowing, loss of viscoelastic properties and the subsequent transfer of compressive loads to the annulus fibrosis. The altered anatomic dimensions and subsequent abnormal response to loading can cause loss of pre-tension in the ligametum flavum, and longitudinal ligaments, abnormal loading and degeneration of the facet capsules (and possible subluxation) with a consequence of secondary degenerative osteoarthritis of the joints.

Spinal disorders requiring neural decompressive surgery can leave motion segment units unstable due to the removal of supporting structures of the joint. A severely unstable motion segment unit is most likely to be fused to ensure post-decompression stability of the joint. The need to fuse the vertebrae of a motion segment unit is dependent on the pre-operative symptoms and clinical (radiographic) findings and on the outcome of the surgical procedure.

One effort at mechanically determining spinal instability is disclosed in “A Technique For Mechanical Assessment Of The Intervertebral Joint), Mark Lubin et al., Biomech. Sym. ADM vol. 43 (1981). A Cloward Lamina Spreader is fitted with a strain gauge and a loading and unloading force is applied manually. The device disclosed in the aforementioned publication is disadvantageous because there is no recognition of the need to control the rate of displacement nor a means for doing so which enables precise measurements of the relative stiffness of the motion segment unit. The motion segment unit is a viscoelastic structure and therefore its resistance to deformation is dependent on the loading rate.

Mark D. Brown and David Holmes (U.S. Pat. No. 4,899,761) disclose an apparatus and method for measuring stability of the motion segment unit of the spine by providing a vertebrae distractor including means for applying a constant rate of increasing force against adjacent vertebrae of a motion segment unit to thereby distract or separate the vertebrae and means for detecting and recording the changes in the resistance to distraction. The apparatus enables a surgeon to quantitatively determine whether the spine has lost its ability to function under physiological loads.

The device disclosed in the '761 patent, while providing useful information regarding the relative stiffness of a motion segment unit of the spine, nonetheless, is problematical because it requires the removal of spinal tissue in order to place the distractor legs of the device in a suitable position for operating the device as shown in FIG. 2 of the reference. In particular, it is often necessary to remove the interspinus ligaments from adjacent vertebrae in order to provide placement of the distractor device in an operable position to measure spinal stiffness. The removal of spinal tissue with this procedure often tends to further contribute to the instability of the motion segment unit. Thus, the surgeon must first further destabilize the motion segment unit before a measurement can be taken and this may have a bearing on the type of implantable spinal assist device that may be used to correct the instability and the degree to which the patient may recover from the spinal surgery.

If instability of the motion segment unit is found, current spinal surgical techniques require the removal of ligaments and bone, in addition to sections of the intervertebral disc. The result of such surgical procedures diminish the structural integrity of the spine joint (i.e. may increase spinal instability). An unstable motion segment unit may be fused to form a permanent or rigid internal fixation of all or part of the intervertebral joints using such materials as rods, hooks, metallic mesh, plates, bolts, screws and/or cement. However, permanent spinal fixation is a difficult surgical technique due to the irregular shape of the bones, the relative weakness of most of the bones of the spine and the complexity and strength of the determining muscular forces acting on the trunk, and more often results in failed back syndrome leaving the patient with persistent pain and debilitating symptoms and/or the need for reoperation.

David C. Holmes (U.S. Pat. No. 5,415,661) discloses an implantable prosthetic device for supporting and reconstructing a motion segment unit of the spine in such a manner that normal or near normal biomechanical function may be restored. Unlike rigid metallic implants whose purpose is to promote fusion of the spine joint, the flexible or compliant spinal implant disclosed in the reference patent is intended to perform as a reconstructive prosthesis allowing for normal or near normal range of motion while supporting the spine joint such that any remaining soft tissue may heal or further damage to that tissue may be prevented. The support provided by the compliant implant in conjunction with the support of the existing soft tissues is intended to replicate the physiological response of a healthy motion segment unit.

While such devices for measuring spinal instability can quantitatively determine the level of instability of the motion segment unit and while implantable devices are available such as disclosed in U.S. Pat. No. 5,415,661, there is no comprehensive method for precisely determining the type of implantable device that is particularly suited for a patient once the quantitative level of instability is measured through devices such as those disclosed in U.S. Pat. No. 4,899,761.

It would therefore be a significant advance in the art of correcting instability of a motion segment unit of the spine if a comprehensive apparatus and method could be developed for matching quantitative levels of instability with particular implantable devices for correcting the instability during a surgical procedure.

In this regard, Applicant has determined that not only are measurements of certain characteristics of spinal instability necessary for the proper determination of a suitable spinal implant device, but that the amount and type of tissue removed during surgery and the location of the same will have a bearing on choosing a suitable spinal implantable device. As such, it is imperative in accordance with the present invention to make use of a data bank of stored information regarding known motion segment units. The stored information can include information regarding relative stiffness, displacement of the motion segment unit at a predetermined force, and hysteresis (energy absorbed by the joint when displaced) to insure that the proper spinal assist device is selected for the patient at critical junctures in the surgical procedure.

### SUMMARY OF THE INVENTION

The present invention is generally directed to an apparatus and method of restoring stability and/or biomechanical function to an unstable motion segment unit of the spine of a patient (hereinafter a “targeted motion segment unit,” or “targeted adjacent vertebrae”) by generating data generally concerning the reaction of the targeted adjacent vertebrae to the application of an applied force (referred to herein as a characteristic of the motion segment unit) and to a comparison of that data with data obtained from a data bank of known motion segment units tested in like manner hereinafter referred to as “sample targeted motion segment unit” or “sample targeted adjacent vertebrae” to provide for the selection of a suitable implantable spinal assist device (ISAD).

The implant selected for restabilization of any one patient's spine joint will be in part, a function of an objective, intraoperative measurement of a characteristic of the motion segment unit based on the application of an applied force (e.g. stiffness), which in turn, is a function of the integrity of a patient's surviving spine joint tissues at this time-post decompression. Measurements of such characteristics that may be employed include relative stiffness which is force per unit distance (e.g. Newtons/mm), displacement of the motion segment unit measured at a predetermined force (e.g. a maximum force at which the adjacent vertebrae do not move further apart [referred to herein as displacement at a predetermined force]) and hysteresis which is the area under a curve formed by the measurement of relative stiffness and a curve formed by the release of the applied force from the motion segment unit and its return to its prior static position.

In a particular aspect of the present invention, there is provided an apparatus and method for restoring stability to an unstable motion segment unit of the spine comprising:

a) applying a force against at least one pair of targeted adjacent vertebrae of a patient;

b) measuring at least one characteristic of the targeted adjacent vertebrae as a function of the applied force and generating an output signal corresponding to the characteristic of said targeted adjacent vertebrae;

c) comparing the output signal to a data bank of values of the same at least one characteristic obtained from sample pairs of targeted adjacent vertebrae tested in the same manner as the targeted adjacent vertebrae, said values of the at least one characteristic of the sample targeted adjacent vertebrae being matched with implantable spinal assist devices capable of at least reducing any instability of the targeted adjacent vertebrae;

d) selecting at least one suitable implantable spinal assist device, if any, from the procedure set forth in steps a), b) and c); and

e) installing the selected implantable spinal assist device in said targeted adjacent vertebrae of the patient to at least reduce any instability to said targeted adjacent vertebrae of said patient.

The steps a), b) and c) may be repeated as often as necessary at different stages of the procedure to analyze and correct spinal instability. For example, the surgeon may apply the force to determine that at least one characteristic of the motion segment unit to constantly monitor loads on the motion segment unit, and to continually provide stiffness, load and/or displacement measurements throughout the surgical procedure including pre decompression, during decompression, post decompression and post implantation.

### BRIEF DESCRIPTION OF THE DRAWINGS

The following drawings are illustrative of embodiments of the invention and are not intended to limit the invention as described in the application.

FIG. 1 is a graph showing the relationship between force and displacement to obtain a measurement as to the relative stiffness of a motion segment unit;

FIG. 2 is a graph showing the relationship between force and displacement in both flexion and extension motions of the motion segment unit and the return of the motion segment unit to its static position to provide a hysteresis measurement;

FIG. 3 is a graph showing a data bank of stiffness values for sample targeted motion segment units; and

FIG. 4 is a graph showing the selection of a spinal assistance device with a predetermined degree of flexibility/stiffness based on the stiffness of a sample targeted motion segment unit.

### DETAILED DESCRIPTION OF THE INVENTION

The present invention is premised on the discovery that there is a relationship between interpretable data obtained from the motion segment unit of a patient's spine (e.g. relative stiffness, displacement at a predetermined force and hysteresis) and a data bank of interpretable data obtained from other motion segments of other spinal columns that can be used to select a suitable implantable spinal assist device (ISAD) which is generally comprised of rigid implantable spinal assist devices (RISAD) and those which are flexible such as those made of composite materials (CISAD) as disclosed in U.S. Pat. No. 5,415,661, and including artificial discs or articulating joints, artificial ligaments, facet capsule replacements, nucleus replacements, and/or annulus replacements, and/or biologics (tissue regeneration technology).

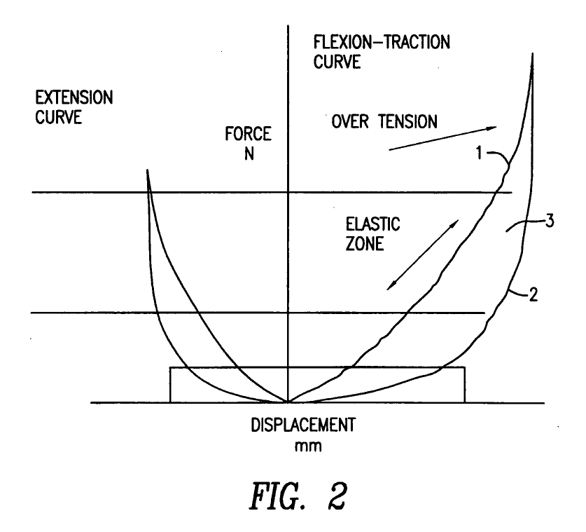

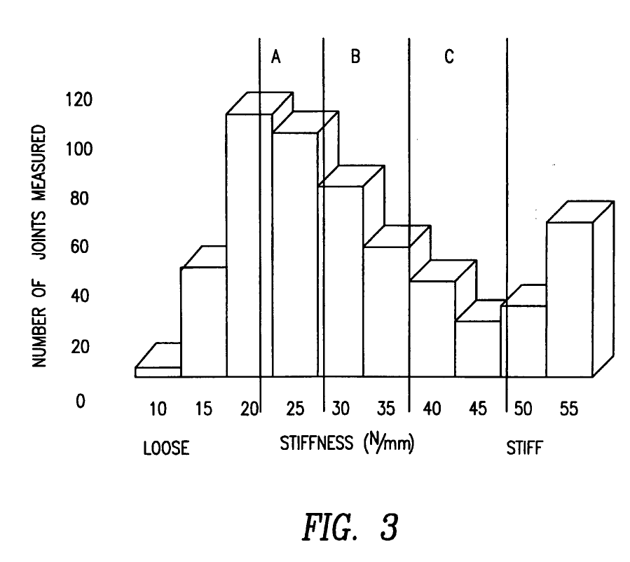

Implantable spinal assist devices of the type described in U.S. Pat. No. 5,415,661 are rendered operational by attachment to the pedicles (posterior surgical approach) or vertebral bodies (lateral and/or anterior surgical approach) of adjacent vertebrae, or a combination thereof. As shown in FIGS. 2 and 3 of the reference patent, holes are provided in the implantable spinal assist device enabling a pedicle screw to be inserted into the pedicle region of a vertebrae.

During surgery, tissue around the vertebrae including tissue in the pedicle region is typically further damaged (the surgical iatrogenic factor) which may well add to the degree of instability of the motion segment unit. Because measurements of instability with devices such as shown in U.S. Pat. No. 4,899,761 and particularly devices in which measurements are made at a constant rate of distraction, are taken prior to the decompressive procedure during surgery, such devices are not always able to take in to account further damage that may occur as a result of the surgical procedure.

Determining the resistance of targeted adjacent vertebrae comprising a motion segment unit to an applied force (e.g. stiffness, displacement at a predetermined force and hysteresis) provides information which can be used in accordance with the present invention to select a suitable implantable spinal assist device particularly matched to the level of instability of the targeted adjacent vertebrae or a series of targeted adjacent vertebrae. Such measurements can be made with instruments such as disclosed in U.S. Pat. No. 4,899,761. However, the information obtained by the use of such devices does not provide a method and comprehensive system for precisely selecting a suitable implantable spinal assist device.

In accordance with the present invention, the data obtained from the measurement of certain characteristics associated with the application of force on the patient's targeted adjacent vertebrae is then compared with a data bank of measurements of the same characteristics from sample targeted motion segment units which may be taken from other patients and/or cadavers over a wide range of values including pre and post decompression motion segment unit measurements.

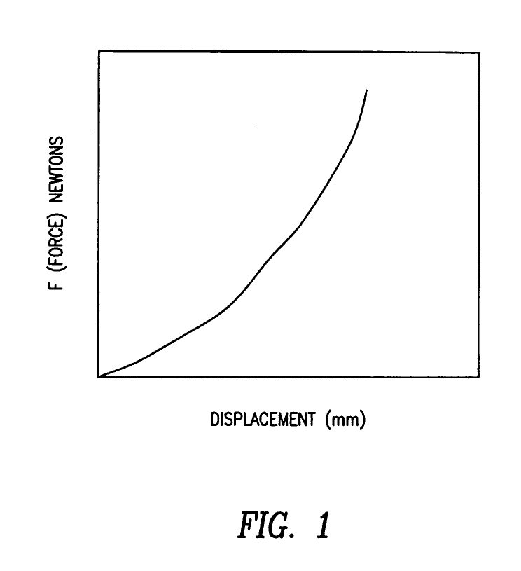

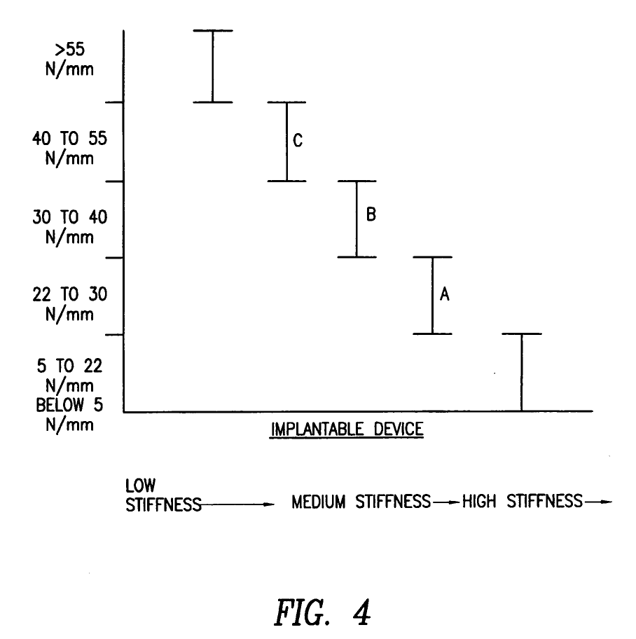

By way of example, the relative stiffness of a spine (i.e. the resistance of a motion segment over distance to a force applied thereto) can be plotted as shown in FIG. 1. The applied force measured in Newtons (y-axis) is plotted against displacement measured in mm (x-axis). As shown in FIG. 1, as the force is applied at a constant or variable rate of distraction, the amount of displacement increases. Therefore, the measured characteristic of the motion segment unit such as stiffness shown in FIG. 1 can be plotted. As shown in FIG. 4, there are, for example, five regions which share stiffness values within a predescribed range. For example, there is a region which identifies a motion segment unit having a relatively low stiffness value of less than 22 N/mm. A suitable approach to correcting the instability associated with this particular targeted motion segment unit would be to use a highly stiff implantable device (e.g. rigid implant) or to replace the motion segment unit. FIG. 4 shows an additional three regions identified as Regions A, B and C in which stiffness values for targeted motion segment units will be matched with appropriate implantable spinal assist devices. It will be understood that more or less than five regions may be defined. Generally, the greater the number of regions, the greater the similarity of stiffness values within a single region.

Again, as shown in FIG. 4, Region A is that area defined by a relatively low force value and high displacement value (e.g. low stiffness) which is indicative of an unstable motion segment unit. Region B lies between Regions A and C and is generally indicative of a motion segment unit that is more stable than Region A, but less stable than Region C, and therefore has a relatively minor degree of instability. Region C showing a relatively high force verses displacement (i.e. high stiffness) is indicative of a relatively rigid motion segment unit, capable of carrying out normal or near normal biomechanical motion of the spine or requires slight adjustment to regain such normal or near normal biomechanical motion. The regions showing stiffness values higher than Region C indicate adjacent vertebrae that may be in a substantially fused condition. Each of these regions of relative stiffness may be matched with a suitable implantable spinal assist device in accordance with the present invention.

When a graph such as that shown in FIG. 3 is generated based on a data bank of a statistically significant samples of targeted motion segment units, it provides significant information useful for the surgeon to ultimately make a more judicious selection of a spinal assist device. In this process, the surgeon can compare data obtained from the targeted motion segment unit to the data obtained from sample motion segment units including during and after decompression where some additional spinal tissue may have been removed as explained hereinafter to facilitate the selection of a spinal assist device.

The data bank also includes information pertaining to the matching of one or more implantable spinal assist devices with the particular stiffness value associated with the sample motion segment units. Such information will typically include an identification of the spinal assist device as applicable to Regions A, B or C shown in FIG. 4 (or greater number of regions as desired) and may include specific information as to the force per displacement values (i.e. stiffness) measured in newtons/mm. In this way, the surgeon can readily match the stiffness value of the targeted motion segment unit with relevant sample motion segment units of the data bank both pre and post surgery to ascertain a suitable selection of an implantable spinal assist device to correct or at least substantially reduce the instability that may be associated with the targeted motion segment unit.

Referring to FIG. 3, there is shown a graph showing exemplary information contained within a data bank relative to sample motion segment units. The y-axis is the number of each group of sample motion segment units tested and the x-axis is the stiffness measured in N/mm associated with each group of sample motion segment units. As indicated in FIG. 3, sample motion segment units summarized therein have a stiffness value ranging from as low as a 5 N/mm up to 65 N/mm and higher (65 N/mm in this case indicates a fused joint) with the lower stiffness values constituting very unstable motion segment units and higher stiffness values be associated with relatively stiff sample motion segment units including those that may actually be fused by surgery or by natural degeneration (i.e. where adjacent vertebrae become locked to each other because of the degeneration of intermediate tissue—also known as functional fusion).

As further indicated in FIG. 3 and by way of example, five categories of stiffness have been identified. The first category of stiffness ranges from about 5 to 22 N/mm, the second category from about 22 to 30 N/mm, the third category from about 30 to 40 N/mm, the fourth category from about 40 to 55 N/mm and the fifth category from 55 N/mm and above.

The stiffness values for the targeted motion segment unit can then be matched with a spinal implant device which will best be suited to provide the spine with required support and to enable the spine to carry out normal biomechanical function.

By way of example only and referring to FIGS. 3 and 4, the second, third and fourth stiffness categories have been designated as Regions A, B and C with each region being associated with a different type of implantable spinal assist device as shown specifically in FIG. 4. By way of example, sample motion segment units having a stiffness value of 22 to 30 N/mm are too loose to support normal or near normal biomechanical function, are matched with, for example, a spinal assist device classified as having relatively high stiffness.

In another example, a sample motion segment unit having a stiffness value in the range of 40 to 55 N/mm (high degree of stiffness) may be matched with an implantable device that is relatively low in stiffness (i.e. Region C shown in FIGS. 3 and 4).

As a result, when the surgeon identifies the relative stiffness of the targeted motion segment, the stiffness value can be compared with sample targeted motion segment units as shown in FIG. 3 and then a suitable spinal implant device can be chosen in accordance with FIG. 4.

It will be understood that comparisons of the relative stiffness of targeted motion segment units can be made at many times during the same surgery, pre-decompression, post decompression, and post implantation, to take into account any change in the relative stiffness values such as may be caused by damage to the surrounding tissues of the targeted motion segment unit, and changes attributed to the then restabilized joint.

While the discussion above exemplifies the present invention by measuring relative stiffness (force per unit distance of distraction) as a characteristic of a target motion segment unit, it will be understood that the present invention can be performed by measuring other characteristics of a motion segment unit based on an applied force such as displacement (mm) at a predetermined force and hysteresis (energy absorbed by the motion segment when displaced and measured in Newtons millimeters [Nmm]) or combinations thereof.

Referring to FIG. 2 there is shown a graph recording the application of force over distance up to a maximum applied force (line 1) and the release of force on the motion segment unit as it returns to its original or static position (line 2). The area defined between lines 1 and 2 (designated by the numeral 3), is a measure of hysteresis or energy absorbed by the targeted motion segment unit. Hysteresis of the targeted motion segment unit can be compared to hysteresis values for sample targeted motion segment units which have been matched with suitable implantable devices as previously described.

Similarly, the relative displacement at a predetermined applied force can also be used as a characteristic of the motion segment unit which can be compared to sample motion segment units as previously described.

Referring again to FIG. 2, the measurements of relative stiffness, displacement at a predetermined force and hysteresis have been taken under flexion (i.e. the force applied to a motion segment unit in a direction corresponding to a forward or bending motion) as well as under extension where the force is applied to move the motion segment unit in the opposite direction. It will be understood that the motion segment unit of a spine is capable of six well known degrees of motion and all such degrees of motion can be tested in accordance with the present invention.

The implantable spinal assist devices which can be employed in accordance with the method of the present invention include, but are not limited to, metal rods and plates of any geometry, composite rods and plates of any geometry, spine joint fusion cages, bone cages, dowels, strips, composite implantable spinal assist devices such as disclosed in U.S. Pat. No. 5,415,661, bone including autologous, bank and/or synthetic bone, bone cement, bone replacement materials such as bone morfogenic protein and the like including both compliant or flexible spinal assist devices and rigid spinal assist devices, and including artificial discs or articulating joints, artificial ligaments, fact capsule replacements, nucleus replacements, and/or annulus replacements, and/or the use of biologics (tissue regeneration technology).

Thus in accordance with the present invention, a targeted motion segment unit of a spine of a patient which is suspected of being unstable is identified and then a characteristic of the targeted motion segment unit (e.g. relative stiffness) is measured by applying a force against the targeted adjacent vertebrae to generate a resistance of the same against the applied force. The force that may be applied against the targeted adjacent vertebrae can be any one or more forces in a direction wherein the motion segment unit undergoes flection, extension, lateral bending and translation or combination thereof. Thus, a force can be applied along the lines of the six degrees of freedom typically associated with a motion segment unit of the spine. Multiple applications of force can be applied in order to detect the resistance of the targeted adjacent vertebrae to the applied force and to generate an output signal (e.g. force per unit length of distraction) corresponding to the resistance of the targeted adjacent vertebrae to the force (e.g. relative stiffness).

The output signal is then compared to the data bank which includes two essential levels of information. The first level of information is measured values of one or more characteristics (e.g. stiffness) associated with sample motion segment units taken prior to decompression, or after decompression, and after implantation during the patient's surgical procedure, and preferably all three measurements. The second, and perhaps the most important level of information, is the identification and association of the measured characteristic (e.g. stiffness) of sample motion segment units with particular implantable spinal assist devices and combinations thereof and the matching of such devices with measurements of the same characteristics of the targeted motion segment unit to therefore select, an implantable spinal assist device capable of correcting or at least improving the instability of the targeted motion segment unit and restoring biomechanical function to the joint or joints.

Once the identification of a suitable implantable spinal assist device is made then the chosen device may be implanted by the spinal surgeon.

The selection and installation of an implantable spinal assist device into the patient will often require the removal of some tissue associated with the targeted adjacent vertebrae. In some cases, the removal of this tissue will reduce stability of the spine (i.e. increase the instability of the spine). Thus, the measured characteristic (e.g. stiffness) determined for the targeted motion segment unit at the beginning of the process may actually change as a result of the surgery necessary to install the implantable spinal assist device. Therefore, it may be necessary to not only compare the measured characteristic of the targeted adjacent vertebrae prior to decompression during surgery, but also to anticipate the result of installing an implantable spinal assist device (i.e. loss of tissue) and to make adjustments in the resistance value or stiffness when selecting a suitable implantable spinal assist device. Thus, the data bank preferably includes sample motion segment units both pre decompression and post decompression during surgery to insure the most suitable selection of a spinal assist device for the particular patient's condition.

By way of example, and referring again to FIG. 3, the measured characteristic (i.e. stiffness) of a targeted motion segment unit may, for example, be 33 N/mm. However, in order properly decompress the patient's spinal cord or nerve roots. It may be necessary to remove an amount of tissue, for example, that reduces the resistance value or stiffness to a value less than 30 N/mm (e.g. 28 N/mm) and may actually result in the targeted adjacent vertebrae moving to a category of greater instability. As shown in FIGS. 3 and 4, the reduction of the stiffness of a targeted motion segment unit from 33 N/mm to 28 N/mm may result in a change of implantable device from one suitable for a Region B device to one suitable for a Region A device.

Accordingly, the present apparatus and method, through the matching of measured characteristics of the targeted adjacent vertebrae with a data bank containing values of the same characteristics of sample motion segment units, preferably both pre and post surgery can provide for the suitable selection implantable spinal assist devices for treating the patient's condition and restoring normal or near normal biomechanical function to the spine. Thus, there is provided a comprehensive system for precisely matching an implantable spinal assist device with a targeted motion segment unit, or units, in a manner which will provide the necessary degree of stability to benefit the patient.

The present method may be employed to measure a particular characteristic (e.g. stiffness) of the targeted motion segment unit prior to decompression of the joint, after removal of tissues necessary to decompress the joint, as well as after the implantable spinal assist device has been installed. By way of example, an intraoperative measurement of resistance of the targeted motion segment unit is done once the incision has been made by posterially, anteriorly or laterally which is referred to as a pre-decompression measurement. Such measurements are used to compare biomechanical joint stability to the patient's diagnosis of the degenerative joint disease and/or soft tissue trauma or injury. These measurements are then compared to data from the sample targeted motion segment units obtained from the data bank to determine where the targeted adjacent vertebrae resistance measurement lies relative to the general population as discussed above in connection with FIG. 3.

By way of example, the measurement of a characteristic (e.g. stiffness) may be made through the use of a spinal stiffness gauge either as described in U.S. Pat. No. 4,899,761 or any other device capable of providing a measurement of resistance versus displacement. The measurement is taken by first subjecting the instrument to a minimum pre-test seating load of, for example, five Newtons (i.e. approximately one pound of force) to a maximum seating load of, for example, approximately 10 Newtons (i.e. approximately two pounds of force). The surgeon then operates the instrument to distract the targeted motion segment unit up to a maximum load of, for example, 112 Newtons (i.e. twenty-five pounds of force).

At the maximum load, the motor within the instrument reverses direction and the instrument legs close and the motion segment unit is returned to a zero load. The test may be run two or more times to ensure repeatability of the measurement. The surgeon then removes the instrument from the spine or may choose to utilize the instrument to assist in the decompressive procedure, for example to gain access to the area of decompression, and to assist in the insertion of any one of the implants, and to constantly monitor the loads on the spine joint. The resistance of the targeted motion segment unit may then be calculated in distraction, maximum displacement and hysteresis (i.e. the energy stored in the targeted motion segment unit and is a function of the area between the distraction curve (force versus displacement as shown in FIGS. 1 and 2).

The procedure described above may be repeated to measure compression, torsional stiffness, translational stiffness or any combination of these measurements.

A suitable implantable spinal assist device or combination thereof is selected by comparing the measurements obtained from the targeted motion segment unit to the resistance values of sample motion segment units and matching implantable spinal assist devices obtained from the data bank as previously described. The motion segment unit resistance measurements are then compared to the database based on patient's with similar diagnosis', age, gender, levels of the spine decompressed and other values which may be selected including pre and post surgical conditions (e.g. the presence or absence of select spinal tissue). The measurements may be presented to the surgeon in the form of a scale, a normal distribution curve or in other ways for providing the surgeon with the necessary information to select a suitable implantable spinal assist device.

What is claimed is:

1. A method of restoring stability to an unstable motion segment unit of a spine comprising:
a) applying a force against at least one pair of targeted adjacent vertebrae of a patient;
b) measuring at least one characteristic of the targeted adjacent vertebrae as a function of the applied force and generating an output signal corresponding to the characteristic of said targeted adjacent vertebrae;
c) comparing the output signal to a data bank of values of the same characteristic obtained from sample pairs of targeted adjacent vertebrae tested in the same manner as the targeted adjacent vertebrae, said characteristic values of the sample targeted adjacent vertebrae being matched with implantable spinal assist devices capable of at least reducing any instability of the targeted adjacent vertebrae;
d) selecting a least one suitable implantable spinal assist device, if any, from the procedure set forth in steps (a), (b) and (c); and
e) installing the selected implantable spinal assist device in said targeted adjacent vertebrae of the patient to at least reduce any instability to said targeted adjacent vertebrae of said patient.

2. The method of claim 1 comprising repeating steps a), b) and c) as necessary up to the selection of the at least one suitable implantable spinal assist device.

3. The method of claim 1 wherein the at least one characteristic is selected from relative stiffness, displacement at a predetermined force, and hysteresis and combinations thereof.

4. The method of claim 3 wherein the predetermined force is the force at which the targeted adjacent vertebrae will not move further apart.

5. The method of claim 1 wherein the at least one characteristic of the targeted adjacent vertebrae is measured while the motion segment unit is undergoing distraction, compression, torsion, rotation, translation and combinations thereof.

6. The method of claim 1 wherein the implantable spinal assist device is selected from rigid implantable spinal assist devices, compliant implantable spinal assist devices and combinations thereof.

7. The method of claim 1 comprising applying the force to the targeted adjacent vertebrae without damaging ligamentous tissue connecting the targeted adjacent vertebrae.

8. The method of claim 2 comprising repeating steps (a), (b) and (c) after the selected spinal assist device has been installed.

9. The method of claim 2 comprising repeating steps (a), (b) and (c) pre decompression and post decompression during the same surgical procedure or ensuing surgical procedures.

10. The method of claim 1 wherein step (d) comprises selecting at least one spinal assist device according to at least one parameter which will impact on the ability of the spinal assist device to render the targeted adjacent vertebrae stable, restoring normal, or near normal, biomechanical function.

11. The method of claim 10 wherein the at least one parameter is selected from the group consisting of size, shape, composition and placement at the site of the targeted adjacent vertebrae.

12. The method of claim 1 comprising applying said force at a constant, increasing rate to the targeted adjacent vertebrae.

13. Apparatus for restoring stability to an unstable motion segment unit of the spine comprising:
a) force applying means for applying a force against at least one pair of targeted adjacent vertebrae of a patient;
b) measuring means for measuring at least one characteristic of the targeted adjacent vertebrae as a function of the applied force and generating an output signal corresponding to the at least one characteristic of said targeted adjacent vertebrae;
c) means for comparing the output signal to a data bank of values of the same characteristic obtained from other pairs of adjacent vertebrae, said characteristic values of the sample targeted adjacent vertebrae being matched with an implantable spinal assist device, if necessary, capable of at least reducing any instability of the targeted adjacent vertebrae; and
d) selection means for selecting a least one suitable implantable spinal device.

14. The apparatus of claim 13 wherein the at least one characteristic is selected from relative stiffness, displacement at a predetermined force, hysteresis and combinations thereof.

15. The apparatus of claim 14 wherein the predetermined force is the force at which the targeted adjacent vertebrae will not move further apart.

16. The system of claim 13 wherein the at least one characteristic of the targeted adjacent vertebrae is measured while the targeted motion segment unit is undergoing distraction, compression, torsion, rotation, translation and combinations thereof.

17. The apparatus of claim 13 wherein the spinal assist device is selected from rigid implantable spinal assist devices and flexible implantable spinal assist devices, and combinations thereof.

18. The apparatus of claim 13 wherein the selection means comprises means for selecting the at least one spinal assist device according to at least one parameter which will impact on the ability of the spinal assist device to render the target motion segment unit stable.

19. The apparatus of claim 18 wherein the at least one parameter is selected from the group consisting of implant size, shape, composition and placement at the site of the targeted adjacent vertebrae.

20. The apparatus of claim 13 wherein the force applying means applies said force at a constant or variable rate.
